(19) United States
(12) Patent Application Publication
(10) Pub. No.: US 2024/0215803 A1
(43) Pub. Date: Jul. 4, 2024

(54) SURGICAL TOOL DEVICE HAVING WIRE HYSTERESIS COMPENSATION FUNCTION AND METHOD FOR CONTROLLING SAME

(71) Applicant: ROEN Surgical, Inc., Daejeon (KR)

(72) Inventors: Dong-Hoon BAEK, Seoul (KR); Young-Hoon NHO, Seoul (KR); Dong Soo KWON, Daejeon (KR)

(73) Assignee: ROEN Surgical, Inc., Daejeon (KR)

(21) Appl. No.: 18/562,175

(22) PCT Filed: May 16, 2022
(86) PCT No.: PCT/KR2022/007005

§ 371 Date: Jan. 4, 2024

(30) Foreign Application Priority Data
May 17, 2021 (KR) 10-2021-0063507

(51) Int. Cl. A61B 1/00; A61B 1/005; A61B 1/045

(52) U.S. Cl. CPC A61B 1/00057; A61B 1/00006; A61B 1/00087; A61B 1/0057; A61B 1/045

(57) ABSTRACT

Proposed are a surgical tool device having a wire hysteresis compensation function and a method for controlling the surgical tool device compensating for hysteresis of a wire. The surgical tool device includes a flexible tube part having a surgical tool disposed in a channel positioned inside the tube part, the flexible tube part being configured such that a position and an orientation of the flexible tube part are changed according to a traction wire, a manipulation unit to which a manipulation command is input so as to control the position and the orientation of the flexible tube part or the surgical tool, and a hysteresis compensation drive control unit provided with a plurality of modes so as to change the position and the orientation of the flexible tube part and the surgical tool and so as to control hysteresis compensation by calibrating hysteresis of the wire.

### TECHNICAL FIELD

The present disclosure relates to a surgical tool device having a wire hysteresis compensation function and a method for controlling the surgical tool device. More particularly, the present disclosure relates to a surgical tool device having a wire hysteresis compensation function and a method for controlling the surgical tool device configured to compensate for a hysteresis of a wire by performing an initial calibration in an idle state.

### BACKGROUND ART

As illustrated in FIG. 1, for an example, a flexible overtube 10 includes a plurality of flexible surgical tools 21 and 22. The flexible surgical tools 21 and 22 (that is, a first surgical tool 21 and a second surgical tool 22) are inserted into and disposed in channels 11 and 12 provided in a body of the overtube, respectively. As an example, a position and an orientation of the first surgical tool 21 may be controlled by being towed by first and second traction wires (not illustrated in the drawings).

As illustrated in FIG. 2, the flexible surgical tool has a hysteresis section. Particularly, the hysteresis section may occur when a wire is stretched due to long-term use of the wire. There is a disadvantage that an overtube or a surgical tool is not accurately controlled in such a hysteresis section.

### DISCLOSURE

### Technical Problem

Accordingly, the present disclosure has been made keeping in mind the above problems occurring in the related art, and an objective of the present disclosure is to provide a surgical tool device having a wire hysteresis compensation function and a method for controlling the surgical tool device configured to perform a calibration mode capable of compensating for stretching of a wire in an idle state, the surgical tool device being configured to calculate a forward direction compensation angle and a reverse direction compensation angle and being configured to perform a hysteresis compensation control in an operation mode by applying the calculated forward direction compensation angle and the calculated reverse direction compensation angle, thereby being capable of accurately controlling a position and an orientation.

However, objectives of the present disclosure are not limited to the aforementioned objective, and other objectives not described above may be evidently understood by those skilled in the art from the following description.

### Technical Solution

In order to realize the objective of the present disclosure, there is provided a surgical tool device having a wire hysteresis compensation function, the surgical tool device including: a flexible tube part having a surgical tool disposed in a channel that is positioned inside the flexible tube part, the flexible tube part being configured such that a position and an orientation of the flexible tube part are changed according to a traction of a traction wire; a manipulation unit to which a manipulation command is input so as to control the position and the orientation of the flexible tube part or to control a position and an orientation of the surgical tool; and a hysteresis compensation drive control unit provided with a plurality of modes so as to change the position and the orientation of the flexible tube part or the position and the orientation of the surgical tool according to the manipulation command of the manipulation unit by using the traction wire, and so as to control hysteresis compensation by, in an idle state, calibrating hysteresis of the wire that is stretched according to use.

In addition, the surgical tool device may further include: a camera part disposed in the channel; and a motor part configured to provide a traction drive force so that the wire is towed according to the manipulation command.

In addition, the plurality of modes may include: a calibration mode in which a forward direction hysteresis and a reverse direction hysteresis of the wire are calibrated in the idle state; and an operation mode in which an initial drive performed so as to leave the idle state of the surgical tool or the flexible tube part is controlled by using a calibration compensation value calculated in the idle state according to drive of the calibration mode, thereby compensating and controlling the hysteresis of the wire.

In addition, the hysteresis compensation drive control unit may include: a mode switching unit configured to switch the plurality of modes; a forward direction compensation mode control unit configured to control the motor part such that the motor part is driven in a first direction in the calibration mode, thereby calculating a forward direction calibration compensation value in the idle state; a reverse direction compensation mode control unit configured to control the motor part such that the motor part is driven in a second direction in the calibration mode, thereby calculating a reverse direction calibration compensation value in the idle state; and an operation mode control unit configured to compensate and control the hysteresis of the wire in an initial drive state by, in the operation mode, applying a total calibration compensation value calculated by using the forward direction calibration compensation value and the reverse direction calibration compensation value in the idle state, thereby controlling the initial drive that is performed so as to leave the idle state.

In addition, each of the forward direction compensation mode control unit and the reverse direction compensation mode control unit may be configured to receive a motor position value in the idle state and a motor position value in a loading state at the time when load is applied by the wire that is stretched and pulled according to the drive of the calibration mode, thereby respectively calculating the forward direction calibration compensation value and the reverse direction calibration compensation value.

In addition, each of the forward direction compensation mode control unit and the reverse direction compensation mode control unit may include: a motor initial position value calculation unit configured to receive the motor position value in the idle state; a motor electric current change check unit configured to determine a time when the stretched wire is pulled and the load is applied on the basis of a change in a motor electric current; a motor final position value calculation unit configured to receive the motor position value in the loading state on the basis of an electric current change check signal of the motor electric current change check unit; and a compensation value calculation unit configured to calculate a hysteresis calibration compensation value of the wire in the idle state on the basis of the motor position value in the idle state and the motor position value in the loading state.

In addition, each of the forward direction compensation mode control unit and the reverse direction compensation mode control unit may be configured to receive a motor position value in the idle state, a pixel value in the idle state input from the camera part in the idle state, and a motor position value in a pixel change state at the time when the wire that is stretched is pulled according to the drive of the calibration mode and then a change in the pixel value input from the camera part occurs, thereby respectively calculating the forward direction calibration compensation value and the reverse direction calibration compensation value.

In addition, each of the forward direction compensation mode control unit and the reverse direction compensation mode control unit may include: a motor initial position value calculation unit configured to receive the motor position value in the idle state; a camera pixel change check unit configured to determine the change in the pixel value by using the pixel value in the idle state and the pixel value at the time when the stretched wire is pulled and load is applied; a motor final position value calculation unit configured to receive the motor position value in the pixel change state on the basis of a pixel change check signal of the camera pixel change check unit; and a compensation value calculation unit configured to calculate a hysteresis calibration compensation value of the wire in the idle state on the basis of the motor position value in the idle state and the motor position value in the pixel change state.

In addition, in order to realize the objective of the present disclosure, there is provided a method for controlling a surgical tool device having a wire hysteresis compensation function, the method including: a process of performing a first direction calibration of a wire by performing a first direction calibration mode in an idle state so that a motor part is controlled such that the motor part is driven in a first direction; a process of calculating a first direction calibration compensation value of the wire in the idle state according to the performance of the first direction calibration; a process of performing a second direction calibration of the wire by, after the performance of the first direction calibration, performing a second direction calibration mode in the idle state so that the motor part is controlled such that the motor part is driven in a second direction; a process of calculating a second direction calibration compensation value of the wire in the idle state according to the performance of the second direction calibration; a process of calculating a total calibration compensation value in the idle state by using the first direction calibration compensation value and the second direction calibration compensation value; and a process of controlling hysteresis of the wire in an initial drive state by performing an operation mode in which an initial drive performed so as to leave the idle state so that the initial drive of a surgical tool or a flexible tube part is controlled by using the total calibration compensation value in the idle state of the wire.

In addition, each of the first direction calibration mode and the second direction calibration mode may include: a process of receiving a motor position value of the motor part in the idle state; and a process of respectively calculating the first direction calibration compensation value and the second direction calibration compensation value by receiving a motor position value in a loading state at the time when load is applied by the wire that is stretched and pulled according to the drive of each calibration mode.

In addition, each of the first direction calibration mode and the second direction calibration mode may include: a process of receiving a motor position value of the motor part in the idle state; a process of receiving a pixel value in the idle state input from a camera part; and a process of respectively calculating the first direction calibration compensation value and the second direction calibration compensation value by receiving a motor position value in a pixel change state at the time when the wire that is stretched is pulled according to the drive of each calibration mode and then a change in the pixel value input from the camera part occurs.

### Advantageous Effects

According to the present disclosure as described above, the forward direction compensation angle and the reverse direction compensation angle (otherwise, the wire length) is calculated by performing the calibration mode capable of compensating for stretching of the wire in the idle state, and the hysteresis compensation control is performed in the operation mode by calculating and applying the total compensation angle (otherwise, the total wire length) by using the calculated forward direction compensation angle and the calculated reverse direction compensation angle, so that there is an effect that accurate position and orientation control is capable of being performed.

In addition, according to the present disclosure, since a separate space sensor or a motion tracking camera is not required to compensate for the hysteresis of the wire, there is an effect that a user's manipulability is capable of being increased while existing equipment is used without any additional equipment.

### DESCRIPTION OF DRAWINGS

The accompanying drawings illustrate exemplary embodiments of the present disclosure, and are provided together with the detailed description for better understanding of the technical idea of the present disclosure. Therefore, the present disclosure should not be construed as being limited to the embodiments set forth in the drawings.

FIG. 1 is a view illustrating a flexible overtube according to an embodiment of the present disclosure,

FIG. 2 is a view illustrating a hysteresis characteristic or a hysteresis curve of a flexible surgical tool according to an embodiment of the present disclosure,

FIG. 3 is a view illustrating a flexible overtube device according to an embodiment of the present disclosure,

FIG. 4 and FIG. 5 are views schematically illustrating a configuration of a surgical tool device having a wire hysteresis compensation function according to an embodiment of the present disclosure, in which FIG. 4 is a view illustrating respectively transmitting an encoder value and a motor electric current value to a forward direction compensation mode control unit and a reverse direction compensation mode control unit and FIG. 5 is a view illustrating respectively transmitting an encoder value and a pixel value to the forward direction compensation mode control unit and the reverse direction compensation mode control unit,

FIG. 6 is a view illustrating calculating a hysteresis compensation value by detecting an electric current change of a motor according to an embodiment of the present disclosure,

FIG. 7 is a view illustrating calculating a hysteresis compensation value by detecting a pixel change of a camera according to an embodiment of the present disclosure, and

FIG. 8 is a view sequentially illustrating a method for controlling a surgical tool device having a wire hysteresis compensation function according to an embodiment of the present disclosure.

### MODE FOR INVENTION

Hereinafter, exemplary embodiments of the present disclosure will be described in detail with reference to the accompanying drawings. However, it will be understood by those skilled in the art that the embodiments described hereinafter does not unfairly limit the contents of the present disclosure described in claims and all components described in the embodiments may not be essential. Moreover, it will be also understood by those skilled in the art that descriptions of conventional arts and matters that are obvious to those skilled in the art can be omitted, and explanation or description of such omitted components (methods) and functions may be sufficiently referred without departing from the technical scope and idea of the present disclosure.

A flexible surgical tool 10 according to an embodiment of the present disclosure includes a device configured as a joint or a flexible tube capable of being towed by a wire, thereby being capable of controlling a position and an orientation thereof. As an example, the flexible surgical tool may include an endoscopic device or an overtube device. Hereinafter, for convenience of description, an overtube device will be described as an example.

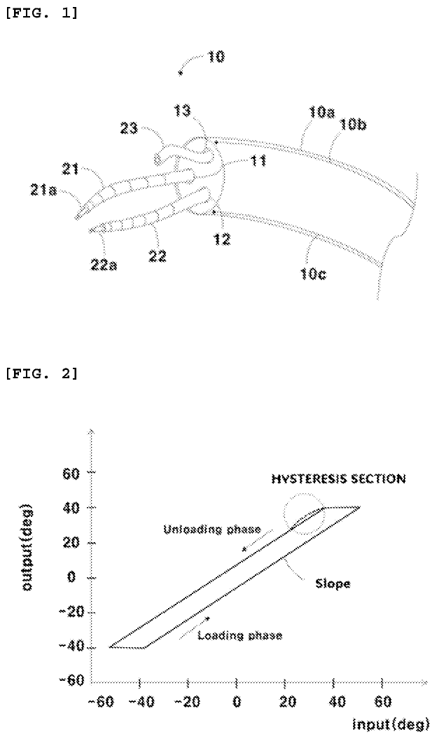

As illustrated in FIG. 1, a wire hysteresis compensation control device of the flexible surgical tool 10 is a device inserted into the human body, and is configured to perform surgery by controlling the position and the orientation of a surgical tool through a traction wire. The flexible surgical tool 10 includes an overtube body 10a having a flexible body, first, second, and third channels 11, 12, and 13 formed such that the first, second, and third channels 11, 12, and 13 have predetermined diameters set inwards of the overtube body 10a, and surgical tools 21, 22, and 23 inserted into each channel 11, 12, and 13. The surgical tools includes tools 21 and 22 or a camera 23 inserted into each channel required for performing surgery.

As illustrated in FIG. 1, a first surgical tool 21 may be inserted into the first channel 11, a second surgical tool 22 may be inserted into the second channel 12, and a camera part 23 may be inserted into the third channel 13. However, the number of channels and the number of surgical tools may be changed as required. The camera part 23 and the surgical tools that are inserted into each channel protrude from a front end portion of the overtube body 10a. Therefore, the camera part 23 may photograph a front side, and each surgical tool may control surgery of pinching or cutting tissue according to a manipulation of the traction wire. A manipulation unit (or a control unit, not illustrated in the drawings) capable of manipulating the traction wire (not illustrated in the drawings) is disposed on a rear end portion of the overtube body 10a. The traction wire is connected from the manipulation unit to the front end portion of the overtube body or a front end portion of the surgical tool. Therefore, the position and the orientation of the surgical tool or the overtube body 10a may be controlled by a user's manipulation of the manipulation unit. At this time, as an example, when a first traction wire 10b and a second traction wire 10c are adjusted by the manipulation unit in order to control the position and the orientation of the flexible overtube body 10a, the position and the orientation of the overtube body 10a are capable of being changed according to a traction force. In addition, in the same manner as the position and orientation control of the flexible overtube body 10a, the position and the orientation of each surgical tool may also be changed according to a traction force of the traction wire. Therefore, although not illustrated in the drawings, each surgical tool is also connected to the traction wire.

Meanwhile, as illustrated in FIG. 2, the flexible overtube body 10a or the flexible surgical tools 21 and 22 have a linear slope section according to the traction force of the traction wire, so that the position and the orientation of each surgical tool are capable of being controlled. However, even when each surgical tool is controlled by using the traction wire, there is a section in which the position and the direction of each surgical tool cannot be controlled consistently corresponding to the traction force even though the same traction force is provided as illustrated in FIG. 2. This section may be referred to as a hysteresis region. Furthermore, in such a hysteresis region, even when the same traction force is applied, the degree of control of the position and the orientation of each surgical tool may vary.

In addition, ideally, the position of the surgical tool should be accurately controlled according to the traction force, so that the graph in FIG. 2 should be a straight line. However, due to hysteresis, the graph has a slope shape. For example, the slope in FIG. 2, illustrating the hysteresis phenomenon, shows a position value of the current tool according to the traction force actually applied by the user when the tool is controlled such that the tool is positioned upward in a constant position and is positioned downward in a constant position repeatedly.

As an example, the reason for the occurrence of such a hysteresis region is that a shape or twist of the flexible surgical tool or the flexible overtube may be changed when the flexible surgical tool or the flexible overtube is inserted into the body, or the aging caused by the use of the traction wire may be the reason.

Meanwhile, in a state in which the wire has stretched due to long-term use of the traction wire, when the traction wire is operated in an idle state, a wire hysteresis occurs as much as the wire is stretched, so that accurate position and orientation control is not capable of being performed even by the manipulation of the manipulation unit.

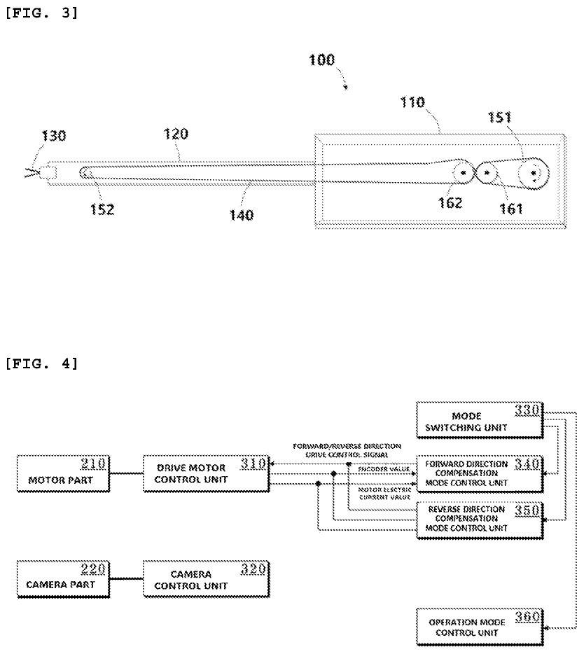

Particularly, as illustrated in FIG. 3, the same problem occurs when a tension of the stretched wire is reduced or increased by using first and second tension adjustment fixing pulley parts 161 and 162.

Therefore, in the present disclosure, a wire calibration is performed in the idle state (a drive stopping state in which the wire is not driven), and a calibration value is applied in an initial driving state in which the wire is driven, so that an accurate position and direction control may be realized in the initial driving state. Hereinafter, a surgical tool device having a wire hysteresis compensation function according to an embodiment of the present disclosure will be described in detail with reference to the accompanying drawings.

### (Configuration and Function of Surgical Tool Device Having Wire Hysteresis Compensation Function)

A surgical tool device having a wire hysteresis compensation function according to an embodiment of the present disclosure is configured to perform a wire calibration in an idle state so that a forward direction calibration compensation value and a reverse direction calibration compensation value (hereinafter, the compensation value may be a compensation angle or a stretched length of the wire) are calculated, and is configured to calculate a total calibration compensation value by using the calculated forward direction calibration compensation value and the reverse direction calibration compensation value. After the wire calibration is performed, in the wire initial driving state, the wire is initially driven by applying the total calibration compensation value calculated according to the forward direction driving and the reverse direction driving cases, so that the hysteresis of the wire may be compensated when the wire is initially driven. At this time, the forward direction driving and the reverse direction driving may be in any one direction of drive pulley parts 151, 152, 161, and 162 illustrated in FIG. 3.

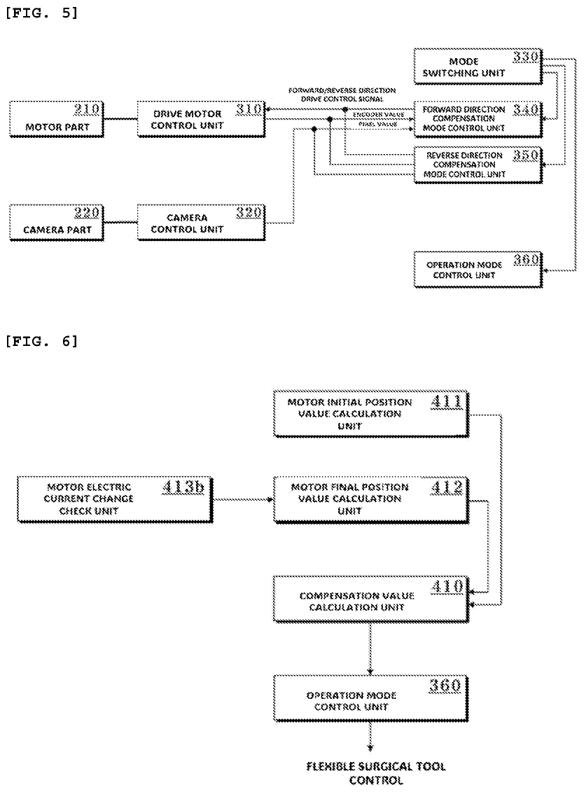

As illustrated in FIG. 4, a motor part 210 according to an embodiment of the present disclosure is configured to rotate the drive pulley parts 151, 152, 161, and 162 in the forward direction and the reverse direction, the drive pulley parts 151, 152, 161, and 162 being configured to tow the wire. At this time, motors configured to drive each of the drive pulley parts 151, 152, 161, and 162 may be respectively disposed. The motor part 210 is configured to drive the drive pulley parts 151, 152, 161, and 162 in the forward direction or the reverse direction according to a control of a drive motor control unit 310. The motor part 210 includes an encoder, and the encoder is configured to measure a rotation angle of the motor. The motor part 210 is configured to drive the drive pulley part according to the control of the drive motor control unit 310, and is configured to transmit an electric pulse signal of the encoder to the drive motor control unit 310 on request. The motor part 210 may be disposed on a drive control unit 110 in FIG. 3.

According to an embodiment of the present disclosure, the position and the orientation of the camera part 220 or 23 may be changed by being towed by the wire according to a control of a camera control unit 320, and the camera part 220 or 23 is configured to transmit a photographed image to the camera control unit 320. The camera control unit 320 is configured to transmit received pixel values to a forward direction compensation mode control unit 340 or a reverse direction compensation mode control unit 350 as illustrated in FIG. 5. At this time, the pixel values transmitted from the camera control unit 320 are an idle state pixel value in the idle state, and are a forward direction motor load pixel value and a reverse direction motor load pixel value in a forward direction and reverse direction calibration mode at the time when the stretched wire is pulled and load is applied to the motor part 210, the forward direction and reverse direction calibration mode being described later. As illustrated in FIG. 1, the camera part 220 or 23 may be disposed in a channel of a flexible overtube 120.

According to an embodiment of the present disclosure, a hysteresis compensation drive control unit includes the drive motor control unit 310, the camera control unit 320, a mode switching unit 330, the forward direction compensation mode control unit 340, the reverse direction compensation mode control unit 350, and an operation mode control unit 360. The hysteresis compensation drive control unit is configured to receive a manipulation command of the manipulation unit, and is configured to control the traction wire according to the received operation command, thereby controlling the position and the orientation of each of the surgical tools 21 and 22 or the camera part 23 or 220. Meanwhile, as the use of the traction wire increases, the traction wire may be stretched as illustrated in FIG. 3. Accordingly, the stretched wire has wire hysteresis characteristics in the idle state. Therefore, the hysteresis compensation drive control unit is provided with a plurality of modes such that the traction wire is calibrated in the idle state and the hysteresis compensation control is performed in the initial drive state. Here, the initial drive state is a state in which the wire leaves the idle state and the wire is initially driven, and the idle state is a state in which the flexible surgical tool is initially driven or a state in which an operation of the flexible surgical tool is stopped for a predetermined time.

Meanwhile, the hysteresis compensation drive control unit performs the plurality of modes, and each mode is switched by the mode switching unit 330. The mode switching unit 330 is configured to control the mode such that the mode is switched according to the manipulation command of the manipulation unit. That is, the mode switching unit 330 is configured to control the mode such that the mode is switched to a forward direction calibration mode when an execution of the forward direction calibration mode is commanded from the manipulation unit, is configured to control the mode such that the mode is switched to a reverse direction calibration mode when an execution of the reverse direction calibration mode is commanded from the manipulation unit, and is configured to control the mode such that the mode is switched to an operation mode when an execution of the operation mode is commanded from the manipulation unit. According to each of the mode execution commands, the forward direction compensation mode control unit 340, the reverse direction compensation mode control unit 350, and the operation mode control unit 360 are respectively executed correspondingly.

The calibration mode is a mode in which a forward direction hysteresis and a reverse direction hysteresis of the wire are calibrated in the idle state. That is, the forward direction hysteresis calibration mode is executed by the forward direction compensation mode control unit 340. The reverse direction hysteresis calibration mode is executed by the reverse direction compensation mode control unit 350. It is preferable that the calibration mode in the other direction is executed after the calibration mode in one direction is executed in the idle state. As an example, the reverse direction calibration mode may be executed after the forward direction calibration mode is executed first. Otherwise, the forward direction calibration mode may be executed after the reverse direction calibration mode is executed first.

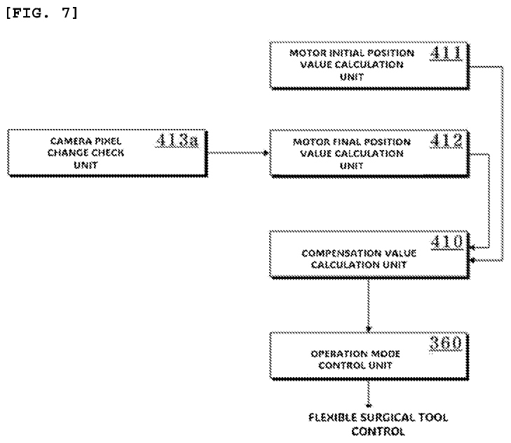

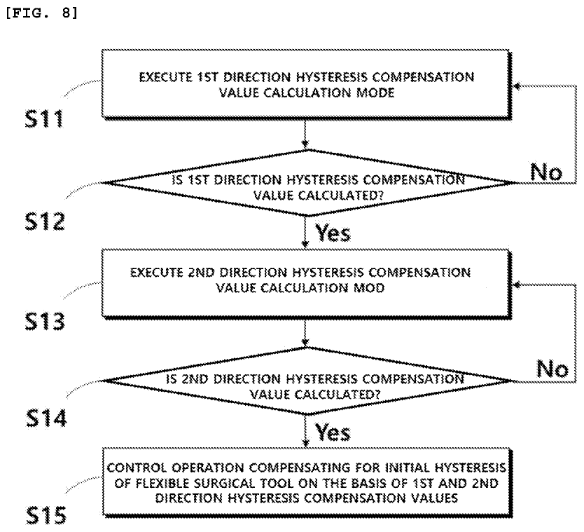

In the forward direction hysteresis calibration mode, the forward direction compensation mode control unit 340 is configured to control the motor part or the drive pulley part such that the motor part or the drive pulley part is driven in the forward direction, thereby calculating the forward direction compensation value in the idle mode. The forward direction calibration compensation value in the idle state may be calculated according to two embodiments as illustrated in FIG. 6 and FIG. 7. However, since the calculation of the reverse direction calibration compensation value may be performed in the same principle as the calculation method of the forward direction calibration compensation value, the description of the calculation of the reverse direction calibration compensation value will be omitted.

In the first embodiment, as illustrated in FIG. 6, the forward direction compensation mode control unit 340 includes a compensation value calculation unit 410, a motor initial position value calculation unit 411, a motor final position value calculation unit 412, and a motor electric current change check unit 413b.

The motor initial position value calculation unit 411 is configured to receive a motor position value in the idle state. The motor final position value calculation unit 412 is configured to receive a motor position value in a loading state at the time when load is applied since the stretched wire is pulled according to an electric current change check signal of the motor electric current change check unit. That is, in the idle state, the motor initial position value calculation unit 411 is configured to receive the motor position value in an initial state in which no load is applied to the motor part 210. In the idle state, the motor final position value calculation unit 412 is configured to receive the motor position value in an initial acting state of a traction force at the time when the stretched wire is pulled and a tension or a traction force occurs. The initial acting state of the traction force is determined according to a setting of the motor electric current change check unit that will be described later.

On the basis of the electric current change of the motor, the motor electric current change check unit 413b is configured to determine the initial acting state of the traction force at the time when the stretched wire in the idle state is pulled and a tension or a traction force occurs. That is, there is no change in the electric current of the motor part 210 in the initial state in which no load is applied to the motor part 210. Then, when the motor part 210 is in the initial acting state of the traction force, the electric current change of the motor part 210 occurs. The motor electric current change check unit 413b is configured to detect the initial acting state of the traction force. Furthermore, when an electric current change detection signal is generated, the motor electric current change check unit 413b transmits the detection signal to the motor final position value calculation unit 412, so that the motor final position value calculation unit 412 calculates the motor final position value in the initial acting state of the traction force.

The compensation value calculation unit 410 calculates the forward direction hysteresis calibration compensation value of the wire in the idle state on the basis of a motor position value in the idle state and a motor position value in the loading state.

In the second embodiment, as illustrated in FIG. 7, the forward direction compensation mode control unit 340 includes the compensation value calculation unit 410, the motor initial position value calculation unit 411, the motor final position value calculation unit 412, and a camera pixel change check unit 413a.

The motor initial position value calculation unit 411 is configured to receive a motor position value in the idle state.

The motor final position value calculation unit 412 is configured to receive a motor position value in a loading state at the time when load is applied since the stretched wire is pulled according to a pixel change check signal of the camera pixel change check unit. That is, in the idle state, the motor initial position value calculation unit 411 is configured to receive the motor position value in an initial state in which no load is applied to the motor part 210. The motor final position value calculation unit 412 is configured to receive the motor position value in an initial acting state of a traction force at the time when the stretched wire in the idle state is pulled and a tension or a traction force occurs and then a change in pixels occurs. The initial acting state of the traction force is determined according to a setting of the camera pixel change check unit 413a that will be described later.

The camera pixel change check unit 413a is configured to receive an initial pixel value in the idle state, the initial pixel value being input from the camera part in the idle state. In addition, the camera pixel change check unit 413a is configured to continuously check whether a change in the pixel value occurs when the initial pixel value is compared with the pixel value at the time when the stretched wire in the idle state is pulled and a tension or a traction force occurs (the initial acting state of the traction force. That is, there is no change in the in the pixel value in the initial state in which no load is applied to the motor part 210. Then, when the motor part 210 is in the initial acting state of the traction force, the direction or the orientation of the camera part 220 is moved, and the change in the pixel value occurs. The camera pixel change check unit 413a is configured to detect the initial acting state of the traction force. Furthermore, when the change in the pixel value is determined by using the pixel value at the time when the stretched wire is pulled and load is applied and the pixel value in the idle state, the camera pixel change check unit 413a generates a pixel change detection signal. The camera pixel change check unit 413a transmits the pixel change detection signal to the motor final position value calculation unit 412, so that the motor final position value calculation unit 412 calculates the motor final position value in the initial acting state of the traction force.

The compensation value calculation unit 410 calculates the forward direction hysteresis calibration compensation value of the wire in the idle state on the basis of a motor position value in the idle state and a motor position value in the loading state.

The reverse direction compensation mode control unit 350 includes the compensation value calculation unit 410, the motor initial position value calculation unit 411, the motor final position value calculation unit 412, the camera pixel change check unit 413a, and the motor electric current change check unit 413b. According to the first embodiment and the second embodiment that are described above, the reverse direction compensation mode control unit 350 may calculate the reverse direction hysteresis calibration compensation value in the same principle as described above by driving and rotating the direction of the drive pulley unit in the reverse direction, and the description of the reverse direction compensation mode control unit 350 will be replaced with the description of the forward direction compensation mode control unit 340 described above.

The operation mode control unit 360 according to an embodiment of the present disclosure is configured to execute the operation mode, and is configured to receive the forward direction calibration compensation value and the reverse direction calibration compensation values in the idle state calculated according to the driving of the forward direction calibration mode and the reverse direction calibration mode. In the operation mode, by applying the total calibration compensation value calculated by using the forward direction calibration compensation value and the reverse direction calibration compensation value in the idle state, the hysteresis of the wire in the initial drive compensated and controlled, so that an initial drive performed so as to leave the idle state is controlled. As an example, when the forward direction calibration compensation angle is 40° and the reverse direction calibration compensation angle is 30°, the total calibration compensation angle is 70°.

### (Method for Controlling Surgical Tool Device Having Wire Hysteresis Compensation Function)

A method for controlling a surgical tool device having a wire hysteresis compensation function of the present disclosure will be described in detail with reference to FIG. 8.

First, the forward direction compensation mode control unit 340 executes the forward direction calibration mode in the idle state, and controls the motor part or the drive pulley unit such that the motor part or the drive pulley unit is driven in the forward direction, thereby executing the forward direction calibration of the wire.

Next, the compensation value calculation unit 410 calculates the forward direction calibration compensation value of the wire in the idle state according to the execution of the forward direction calibration.

Next, after the forward direction calibration mode is executed in the idle state, the reverse direction compensation mode control unit 350 executes the reverse direction calibration mode, and controls the motor part such that the motor part is driven in the reverse direction, thereby executing the reverse direction calibration of the wire.

Next, the compensation value calculation unit 410 calculates the reverse direction calibration compensation value of the wire in the idle state according to the execution of the reverse direction calibration.

Next, the compensation value calculation unit 410 calculates the total calibration compensation value by using the forward direction calibration compensation value and the reverse direction calibration compensation value.

Next, the operation mode control unit 360 executes the operation mode in which the initial drive performed so as to leave the idle state is controlled, and controls the initial drive of the surgical tool or the flexible tube part by using the total calibration compensation value of the wire, thereby compensating and controlling the hysteresis of the wire in the initial drive state.

Meanwhile, as described above, the order of executing the forward direction calibration and the reverse direction calibration is not limited thereto, and the reverse direction calibration may be performed first and then forward direction calibration may be performed.

In the description of the present disclosure, it will be also understood by those skilled in the art that descriptions of conventional arts and matters that are obvious to those skilled in the art can be omitted, and explanation or description of such omitted components (methods) and functions may be sufficiently referred without departing from the technical scope and idea of the present disclosure. In addition, it will be also understood by those skilled in the art that the components described above are provided just for the sake of convenient description and other components which are not described may be added within the range of the technical idea of the present disclosure.

Moreover, it will be also understood by those skilled in the art that the components and functions of the parts are described separately, but any one of the components and functions may be integrated with another component or may be subdivided.

While the present disclosure has been described with reference to the embodiments thereof, it will be appreciated that the present disclosure is not limited thereto, and various changes, modifications and equivalents may be made in the present disclosure. That is, it will be understood by those skilled in the art that various changes and modifications of the present disclosure are possible without departing from the technical scope and idea of the present disclosure. In addition, when it is judged that detailed descriptions of known functions or structures related with the present disclosure or detailed descriptions of combination relations of components of the present disclosure may make the essential points vague, the detailed descriptions of the known functions or structures will be omitted.

1. A surgical tool device having a wire hysteresis compensation function, the surgical tool device comprising:
a flexible tube part having a surgical tool disposed in a channel that is positioned inside the flexible tube part, the flexible tube part being configured such that a position and an orientation of the flexible tube part are changed according to a traction of a traction wire;
a manipulation unit to which a manipulation command is input so as to control the position and the orientation of the flexible tube part or to control a position and an orientation of the surgical tool; and
a hysteresis compensation drive control unit provided with a plurality of modes so as to change the position and the orientation of the flexible tube part or the position and the orientation of the surgical tool according to the manipulation command of the manipulation unit by using the traction wire, and so as to control hysteresis compensation by, in an idle state, calibrating hysteresis of the wire that is stretched according to use.

2. The surgical tool device of claim 1, further comprising:
a camera part disposed in the channel; and
a motor part configured to provide a traction drive force so that the wire is towed according to the manipulation command.

3. The surgical tool device of claim 2, wherein the plurality of modes comprises:
a calibration mode in which a forward direction hysteresis and a reverse direction hysteresis of the wire are calibrated in the idle state; and
an operation mode in which an initial drive performed so as to leave the idle state of the surgical tool or the flexible tube part is controlled by using a calibration compensation value calculated in the idle state according to drive of the calibration mode, thereby compensating and controlling the hysteresis of the wire.

4. The surgical tool device of claim 3, wherein the hysteresis compensation drive control unit comprises:
a mode switching unit configured to switch the plurality of modes;
a forward direction compensation mode control unit configured to control the motor part such that the motor part is driven in a first direction in the calibration mode, thereby calculating a forward direction calibration compensation value in the idle state;
a reverse direction compensation mode control unit configured to control the motor part such that the motor part is driven in a second direction in the calibration mode, thereby calculating a reverse direction calibration compensation value in the idle state; and
an operation mode control unit configured to compensate and control the hysteresis of the wire in an initial drive state by, in the operation mode, applying a total calibration compensation value calculated by using the forward direction calibration compensation value and the reverse direction calibration compensation value in the idle state, thereby controlling the initial drive that is performed so as to leave the idle state.

5. The surgical tool device of claim 4, wherein each of the forward direction compensation mode control unit and the reverse direction compensation mode control unit is configured to receive a motor position value in the idle state and a motor position value in a loading state at a time when load is applied by the wire that is stretched and pulled according to the drive of the calibration mode, thereby respectively calculating the forward direction calibration compensation value and the reverse direction calibration compensation value.

6. The surgical tool device of claim 5, wherein each of the forward direction compensation mode control unit and the reverse direction compensation mode control unit comprises:
a motor initial position value calculation unit configured to receive the motor position value in the idle state;
a motor electric current change check unit configured to determine a time when the stretched wire is pulled and the load is applied on the basis of a change in a motor electric current;
a motor final position value calculation unit configured to receive the motor position value in the loading state on the basis of an electric current change check signal of the motor electric current change check unit; and
a compensation value calculation unit configured to calculate a hysteresis calibration compensation value of the wire in the idle state on the basis of the motor position value in the idle state and the motor position value in the loading state.

7. The surgical tool device of claim 4, wherein each of the forward direction compensation mode control unit and the reverse direction compensation mode control unit is configured to receive a motor position value in the idle state, a pixel value in the idle state input from the camera part in the idle state, and a motor position value in a pixel change state at a time when the wire that is stretched is pulled according to the drive of the calibration mode and then a change in the pixel value input from the camera part occurs, thereby respectively calculating the forward direction calibration compensation value and the reverse direction calibration compensation value.

8. The surgical tool device of claim 7, wherein each of the forward direction compensation mode control unit and the reverse direction compensation mode control unit comprises:
a motor initial position value calculation unit configured to receive the motor position value in the idle state;
a camera pixel change check unit configured to determine the change in the pixel value by using the pixel value in the idle state and the pixel value at the time when the stretched wire is pulled and load is applied;
a motor final position value calculation unit configured to receive the motor position value in the pixel change state on the basis of a pixel change check signal of the camera pixel change check unit; and
a compensation value calculation unit configured to calculate a hysteresis calibration compensation value of the wire in the idle state on the basis of the motor position value in the idle state and the motor position value in the pixel change state.

9. A method for controlling a surgical tool device having a wire hysteresis compensation function, the method comprising:
a process of performing a first direction calibration of a wire by performing a first direction calibration mode in an idle state so that a motor part is controlled such that the motor part is driven in a first direction;
a process of calculating a first direction calibration compensation value of the wire in the idle state according to the performance of the first direction calibration;
a process of performing a second direction calibration of the wire by, after the performance of the first direction calibration, performing a second direction calibration mode in the idle state so that the motor part is controlled such that the motor part is driven in a second direction;
a process of calculating a second direction calibration compensation value of the wire in the idle state according to the performance of the second direction calibration;
a process of calculating a total calibration compensation value in the idle state by using the first direction calibration compensation value and the second direction calibration compensation value; and
a process of controlling hysteresis of the wire in an initial drive state by performing an operation mode in which an initial drive performed so as to leave the idle state so that the initial drive of a surgical tool or a flexible tube part is controlled by using the total calibration compensation value in the idle state of the wire.

10. The method of claim 9, wherein each of the first direction calibration mode and the second direction calibration mode comprises:
a process of receiving a motor position value of the motor part in the idle state; and
a process of respectively calculating the first direction calibration compensation value and the second direction calibration compensation value by receiving a motor position value in a loading state at a time when load is applied by the wire that is stretched and pulled according to the drive of each calibration mode.

11. The method of claim 9, wherein each of the first direction calibration mode and the second direction calibration mode comprises:
a process of receiving a motor position value of the motor part in the idle state;
a process of receiving a pixel value in the idle state input from a camera part; and
a process of respectively calculating the first direction calibration compensation value and the second direction calibration compensation value by receiving a motor position value in a pixel change state at a time when the wire that is stretched is pulled according to the drive of each calibration mode and then a change in the pixel value input from the camera part occurs.
